(19) United States
(12) Patent Application Publication
(10) Pub. No.: US 2018/0310847 A1
(43) Pub. Date: Nov. 1, 2018

(54) WEARABLE MONITORING DEVICE

(71) Applicant: VIRGINIA COMMONWEALTH UNIVERSITY, Richmond, VA (US)

(72) Inventors: Joseph P. Ornato, Richmond, VA (US); Erdem Topsakal, Richmond, VA (US); Marcus C. Massok, West Point, VA (US); Justin A. Artis, Richmond, VA (US); Ashraf AlGumaei, Richmond, VA (US); Jon Dyke, Richmond, VA (US); Majid AlAshari, Richmond, VA (US); Wesley N. Bosman, Richmond, VA (US)

(21) Appl. No.: 15/963,409

(22) Filed: Apr. 26, 2018

Related U.S. Application Data
(60) Provisional application No. 62/490,888, filed on Apr. 27, 2017.

(51) Int. Cl. A61B 5/024; A61B 5/00

(52) U.S. Cl. CPC A61B 5/02427; A61B 5/6824; A61B 2562/0219; A61B 5/0004; A61B 2562/04; A61B 5/747

(57) ABSTRACT

A wearable monitoring device is provided to detect the physiological condition of a user and alert emergency services if needed. As provided herein, the monitoring device may include a band configured to be worn on a user's wrist; two or more operably connected PPG sensors arranged on the band, wherein at least two of the two or more PPG sensors are arranged such that the sensors contact opposite sides of the user's wrist when the device is being worn and wherein the at least two PPG sensors provide a higher signal to noise ratio as compared to a single PPG sensor; and a processor configured to receive and process readings from the at least two PPG sensors. Methods of using the monitoring device are also provided.

### CROSS-REFERENCE TO RELATED APPLICATIONS

This application claims benefit of U.S. provisional patent application 62/490,888, filed Apr. 27, 2017, the complete contents of which is hereby incorporated by reference.

### FIELD OF THE INVENTION

Embodiments of the invention relate to the field of wearable life monitors.

### BACKGROUND OF THE INVENTION

According to the American Heart Association, Cardiac Disease is the most prominent cause of death globally. In America alone 360,000 people annually experience a sudden, unexpected out-of-hospital cardiac arrest (OHCA, or stoppage of the heart's pumping function, causing immediate unconsciousness and loss of a pulse) (American Heart Association, 2018). Every minute that goes by until the heart can be restarted with cardiopulmonary resuscitation (CPR) and defibrillation (shocking the heart to attempt to restore a normal rhythm), the risk of survival drops 10%. Thus, the time interval between the onset of a cardiac arrest and effective medical treatment is critical and unforgiving.

There are approximately 360,000 cases of OHCA every year in the United States (American Heart Association, 2018). In cases in which there is a bystander who witnesses the cardiac arrest and they call 911 immediately (preferably also starting CPR) with a rapid response by trained and equipped emergency rescuers, nationwide survival averages 10% (AHA, 2018). However, only half of cardiac arrest cases are witnessed, and the victim is later found deceased. Thus, the actual survival from cardiac arrest is 5% (1 in 20). Thus, there is an urgent need in the art for a device in which individuals experiencing cardiac events are able to notify emergency responders remotely and without any effort.

### SUMMARY OF THE INVENTION

An aspect of the invention provides an unobtrusive, wearable, affordable device that can perform useful every day functions (i.e., like a watch that can run APPS) but which also accurately tracks the presence of the individual's pulse and automatically contacts 911 with the unconscious patient's location and other vital information (after appropriate safeguards noted herein to ensure the rarity of a false alarm) in the event of a cardiac arrest. This device is of great public health importance if a large segment of the population would wear one in place of a digital watch or fitness band by: 1) allowing immediate detection and 911 notification of a cardiac arrest in the 50% of cases in which the event is unwitnessed; and 2) resulting in 911 dispatch of emergency personnel to the exact patient location within approximately 30 s of the event onset.

The monitoring device, according to embodiments of the invention, is a wrist-wearable device that gathers physiological data of a user. For example, the device may detect cardiac arrest in the user and subsequently alert emergency services of the user's condition and location.

In one aspect of the invention, the monitoring device comprises a band configured to be worn on a user's wrist; two or more operably connected photoplethysmogram (PPG) sensors arranged on the band, wherein at least two of the two or more PPG sensors are arranged such that the sensors contact opposite sides of the user's wrist when the device is being worn and wherein the two or more PPG sensors provide a higher signal to noise ratio as compared to any of the two or more PPG sensors individually; and a processor configured to receive and process readings from the at least two PPG sensors.

In some embodiments, the monitoring device may further comprise an inertial measurement unit (IMU), wherein the processor processes the PPG readings in conjunction with the IMU. In some embodiments, the monitoring device may further comprise one or more bandpass filters, wherein sensor information is passed through the one or more bandpass filters before being received by the processor. In some embodiments, the band is elastic. In further embodiments, the wires connecting the at least two PPG are arranged on the band in a sinusoidal pattern. In some embodiments, the processor is configured to transmit readings from the at least two PPG sensors to a mobile device. The monitoring device may comprise one or more transmitters, receivers, transceivers, or some combination thereof for communicating with external devices or networks. In some embodiments, the processor is configured to transmit readings from the at least two PPG sensors to emergency services.

Another aspect of the invention provides a method of providing assistance to a subject based on a determined physiological condition of the subject, comprising determining that a physiological condition of the subject is abnormal based on information from two or more PPG sensors of a monitoring device worn on a wrist of the subject; and transmitting an alert to emergency services indicating the abnormal physiological condition of the subject and a determined location of the subject.

In some embodiments, the physiological condition is the subject's heart rate. In some embodiments, the alert is transmitted from the monitoring device via a mobile device. In some embodiments, the monitoring device directly transmits the alert to emergency services. In some embodiments, transmitting an alert to emergency services further comprises: upon determining the abnormal condition, the monitoring device prompting the subject and waiting for a response therefrom; and if the subject does not respond to the prompt within a predetermined amount of time, the monitoring device transmitting the alert to emergency services. In some embodiments, determining that a physiological condition of the subject is abnormal further comprises: continually monitoring a physiological condition to determine a value for a normal condition; and provided the physiological condition value varies from the normal condition more than a predetermined amount, generating the alert.

Additional features and advantages of the invention will be set forth in the description below, and in part will be apparent from the description, or may be learned by practice of the invention. The advantages of the invention can be realized and attained by the exemplary embodiments particularly pointed out in the written description and claims hereof as well as the appended drawings.

### BRIEF DESCRIPTION OF THE DRAWINGS

FIGS. 1A and B show alternate views of an exemplary monitoring device according to some embodiments of the disclosure;

FIG. 2 shows exemplary components of an exemplary monitoring device according to some embodiments of the disclosure

FIG. 3 is an exemplary monitoring device according to some embodiments of the disclosure;

FIG. 4 is an exemplary monitoring device according to some embodiments of the disclosure;

FIG. 5 is an exemplary system according to some embodiments of the disclosure; and

FIG. 6 is an exemplary method according to some embodiments of the disclosure.

### DETAILED DESCRIPTION

Embodiments of the invention provide a monitoring device for monitoring physiological parameters such as heart rate, oxygen saturation, breathing, blood volume, body temperature, perspiration, and motion activity (e.g., fall detection).

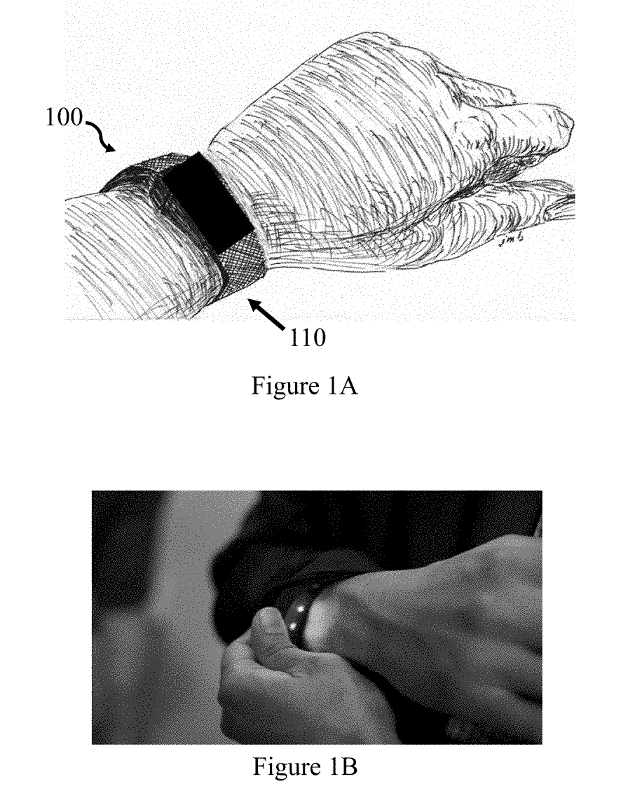

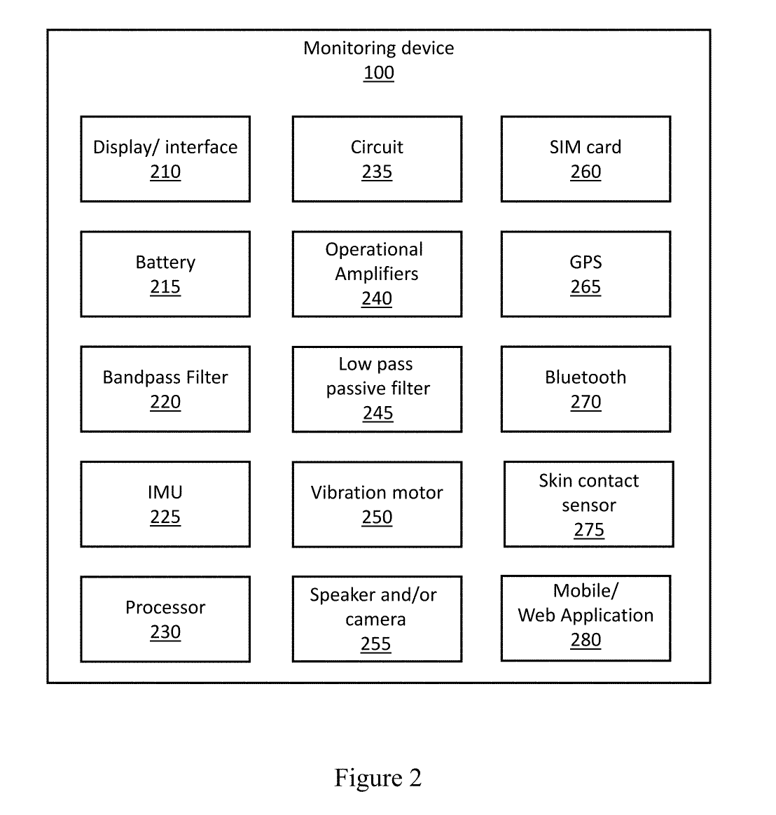

With reference to FIGS. 1A and 1B, the monitoring device 100 comprises a band 110 configured to be worn on a user's wrist. The band 110 may be made of any material suitable to be worn by a user, e.g. plastic, rubber, an elastic or inelastic fabric or material, etc. In some embodiments, the band 110 incorporates a clasp or other type of known closure means that may or may not be adjustable. In other embodiments, the band 110 is manufactured as one-piece of an elastic material and thus the user would slide the band 110 over the hand to the wrist for use. In some embodiments, the monitoring device 100 is a digital “smart watch” which include features in addition to monitoring physiological parameters, such as applications for receiving and sending voice calls and/or text messages and an accelerometer for detecting steps taken while walking or running. In some embodiments, the monitoring device 100 detects one or more physiological parameters without the additional features of a smart watch. With reference to FIG. 2, a digital display (e.g. LCD display) or touchscreen interface 210 may display the one or more physiological parameters being measured and additional information such as the date and time. A rechargeable battery 215 may be used to power the device.

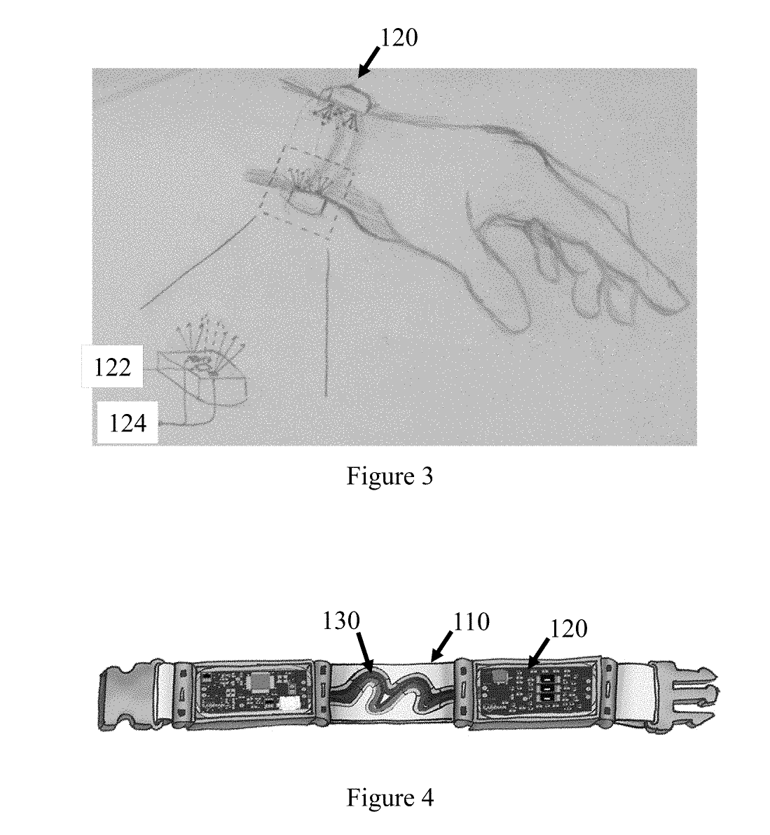

With reference to FIG. 3, two or more (e.g., two, three, four, or more) operably connected PPG sensors 120 may be arranged on the band. “Operably connected” refers to a communicative connection between the sensors, whether through wires or wirelessly. In some embodiments, two of the at least two PPG sensors 120 are arranged such that the sensors contact opposite sides of the user's wrist when the device is being worn. For instance, the spacing of the two sensors may be half or approximately half of the circumference of an average adult human wrist. At least one sensor may be configured to have an adjustable position to accommodate variations in the wrist sizes of individual users, thereby ensuring that each of the at least two sensors can be arranged on opposite sides of the user's wrist in a state of use. Opposite sides of a wrist may mean that a first distance and second distance are substantially the same, where the first distance is the distance from a first sensor to a second sensor clockwise about the wrist or band, and where the second distance is the distance from the first sensor to second sensor counterclockwise about the wrist or band. The first and second distances may vary within accepted tolerances, e.g., the first distance is 0.5 to 2 times the second distance. The first distance may be at least 0.5, 0.6, 0.7, 0.8, 0.9, or 1.0 times the second distance. The first distance may be no larger than 2.0, 1.9, 1.8, 1.7, 1.6, 1.5, 1.4, 1.3, 1.2, or 1.1 times the second distance. The first distance may be specified according to a tolerance range with respect to the second distance: e.g. the first distance is 0.6-1.9 times the second distance, or 0.7-1.8 times the second distance, or 0.8-1.7 times the second distance, or 0.9-1.6 times the second distance, or 1.0-1.5 times the second distance. The distances may be measured as arc lengths. Opposite sides of a wrist may mean a line drawn from the center of photodiode 122 of a first sensor to a center of a photodiode 122 of a second sensor passes through a center of a user's wrist in worn state.

The PPG sensors 120 are arranged on the band with respect to a user interface 210 to reduce user error when installing the device 100 on the wrist. Users familiar with traditional watches frequently position a user interface at the top of the wrist or at the bottom of the wrist, but rarely at other positions about the wrist (e.g., the left side or right side of the wrist). The sensors 120 are positioned on the band with respect to the user interface (e.g., display) 210 such that the sensors 120 are respectively at a top and bottom of the wrist whenever the user interface 210 is positioned at the top or bottom of the wrist in a state of use.

The PPG sensors 120 may include a photodiode 122 and light-emitting diodes (LEDs) 124 to illuminate the skin and measure changes in light absorption. Providing at least two PPG sensors 120 allows for a higher signal to noise ratio as compared to any of the at least two PPG sensors individually. Thus, the number and placement of the sensors allows the device to obtain a noise resistant, motion artifact resistant reading and to more accurately measure heart rate. The dual two PPG sensor (four sensors in total) handle the heart rate signal in a more accurately. Every two sensors will take the data from either the top or the bottom of the wrist, then combine the data from the other side, average the data and get the most accurate heart rate signal. The dual sensor technology can handle the data better than one sensor since it ensures that the data taken from the wrist is not affected by any outside factor such as skin thickness or hair. This allows the data to be read from the top and bottom of the wrist; making the data more precise. To further reduce noise outside the bandwidth of the signal to be measured, the PPG sensor 120 outputs may be passed through a bandpass filter 220. In some embodiments, the sensors may be coupled with an inertial measurement unit (IMU) 225, such as an Arduino IMU.

Output from the sensors and the IMU may be passed to a processor or controller 230, such as a microcontroller, e.g. an ATmeg328 Processor or cortex-M4 processor, where the sensor data and the motion data are used to further reduce motion artifacts. The higher resistance to motion artifacting may be in the directions normal to the face of the sensors. Thus, the processor 230 may be configured to receive and process readings from the at least two PPG sensors 120.

In some embodiments, the device includes an integrated circuit 235 comprising at least two operational amplifiers 240 at low power. In some embodiments, the device includes an LM358 8-pin integrated circuit comprising two operational amplifiers, e.g. a photodiode amplifier and a 1000× amplifier, at low power. The device may also include a low pass passive filter 245. The photodiode(s) 122, LED(s) 124, and processor 230 may be mounted to the integrated circuit 235.

In some embodiments, the sensor configuration described herein may be incorporated into existing smart watches, fitness trackers, health monitors, hospital SPO2 readings, and any other applications where measurement of blood flow and blood oxygen is useful.

With reference to FIG. 4, the PPG sensors 120 may be connected by wires 130 integrated into a stretchable band 110. Wave patterning may be used to allow for wire elasticity, i.e. the wires 130 are incorporated with the elastic band 110 in a waveform (either intermeshed with the elastic band structure or operably affixed to at least one face of the elastic band), such as a sine waveform, such that the effective length of the wires 130 can elongate when the elastic is stretched while the height or amplitude of the wave pattern decreases. The opposite effect is produced upon relaxing of the stretch. This configuration allows the placement of multiple components within a closed loop environment. In alternative embodiments, the PPG sensors 120 are wirelessly connected.

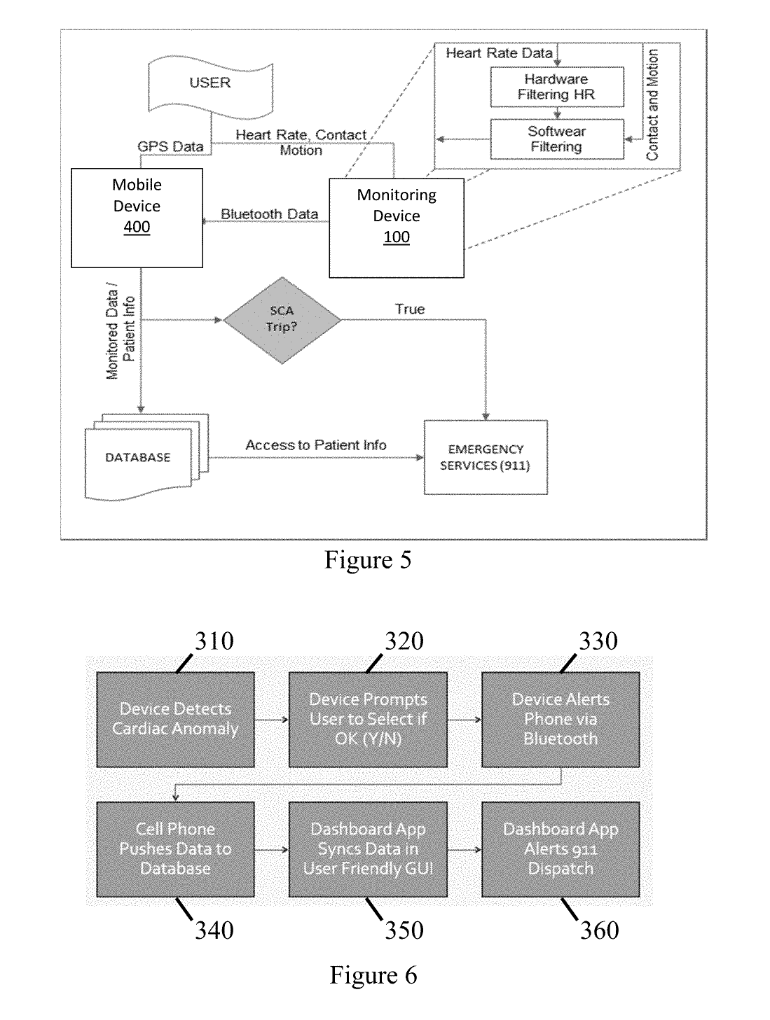

With reference to FIGS. 5 and 6, the monitoring device 100 continually monitors the user's heart activity, e.g., heart rate, via PPG and detects when the user's pulse goes beyond pre-defined cardiac thresholds (e.g. below 20-30 bpm, exceeding 80-90% of the user's maximum heart rate as determined by age and sex or the user's normal activity, lack of pulse, or any other known threshold for detecting abnormalities such as cardiac arrest). If an abnormal cardiac event is detected, the monitoring device 100 may alert the user, e.g. via vibration motor 250 and/or audible signal from a speaker 255 to confirm if they are having an incident, e.g. via a touchscreen 210. If the user confirms an incident is taking place, the watch system may use, e.g. a SIM card 260 to place a call via cellular network(s) to communicate with the emergency services, e.g. the fire department, and provide the user's location (e.g., via GPS 265) and details of the emergency. In some embodiments, a linked cell phone (e.g., via Bluetooth 270) may contact emergency services and provide the location. If the user indicates that an incident is not taking place, an alert is not transmitted. The monitoring device may include one or more skin contact sensor 275 to determine if the device is being worn thus avoiding false alarms. In some embodiments, the PPG light sensor 120 is used to determine if the device is being worn. In alternative embodiments, the skin contact sensor 275 is separate from the PPG sensor 120, e.g. a capacitive skin contact sensor. In some embodiments, the user has the option of initiating the alert themselves even if the device does not detect a cardiac event.

In some embodiments, the alert is transmitted to pre-set emergency contacts such as a 911 center. The emergency contacts may be sent an email and/or text message in which they will be given a unique code that will allow them to view the current location of the device wearer through the respondent side of the web application 280. In some embodiments, the alert is transmitted to a 911 call center. A call center representative may then confirm the emergency and dispatch emergency services and/or contact pre-set emergency contacts.

A web-based application 280 on the monitoring device or a user's mobile device 400, e.g. a cell phone, smartphone, smartwatch, tablet, or laptop computer, may be used to store, retrieve, and update the user's information. The user may add information to the application 280, such as emergency contacts (e.g. friends and family) and medical history, including medical conditions, allergies, medications, and the like.

Some embodiments of the invention provide a method of providing assistance to a subject based on a determined physiological condition of the subject, comprising determining that a physiological condition (e.g. the heart rate) of the subject is abnormal based on information from two or more PPG sensors of a monitoring device worn on a wrist of the subject; and transmitting an alert to emergency services indicating the abnormal physiological condition of the subject and a determined location of the subject.

To accomplish such a method, an initial step is to confirm the monitoring device is being worn e.g. via a skin contact sensor. Following a confirmation that the device is being worn and upon detection of a cardiac abnormality at block 310 (e.g. by measuring a value that extends beyond a predetermined threshold or that varies from the normal condition value of the user by more than a predetermined amount, e.g. by more than 50-60% or more), generating the alert, the device system activates a vibration motor to create a haptic alert and/or an audible signal. At block 320, the touch screen interface may then activate and present (display to) the user with a binary dialog or display queue to confirm there is an emergency, e.g. “Are you having an emergency?” The user is presented with two choices, e.g. “Yes” or “No” to confirm the emergency. The user's selection to cancel or non-confirmation of emergency will terminate the process. After a predetermined threshold of time, e.g. 20-25 seconds, non-response to this prompt (or any) will be considered a confirmation of emergency. At block 330, once the emergency has been confirmed, a SIM card and cellular antenna (within the device or within a connected mobile device) may transmit the wearer's GUID, location, and heart rate data to emergency services. The user will still have the option of canceling the alert after the process of contacting emergency services has begun. At block 340, the monitoring device or user's mobile device may display the user's vitals in real time while also transmitting the data to a database. At blocks 350 and 360, the information in the database which may include vitals, location, medical history, etc. could then be pushed to a dashboard system that provides the nature of the user's emergency to 911 dispatch operators (e.g. Department of Emergency Communications or Medical Dispatch). The direct connection to emergency services cuts down on the lag-time between alert and response for the user. Accordingly, the emergency services system may have a corresponding web application that allows for connection to users of the monitoring device and the user's information. This information can be used to prepare medics for arriving on scene as well as for hospitals to have a plan for patient receiving.

In some embodiments, the initiation of an alert will activate a speaker and/or camera 255 in the monitoring device or mobile device so that 911 paramedic dispatchers can quickly assess the user's condition, e.g. by asking the user “are you ok?” and otherwise see and/or hear what is going on. If there are any bystanders at the user's side, the dispatcher would be able to talk them through doing chest compressions or other techniques until emergency crews arrive.

In some embodiments, the monitoring device is used for remote patient monitoring by doctors both in and out of a hospital.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A monitoring device, comprising:
a band configured to be worn on a user's wrist;
two or more operably connected photoplethysmogram (PPG) sensors arranged on the band, wherein at least two of the two or more PPG sensors are arranged such that the at least two sensors contact opposite sides of the user's wrist when the device is being worn, and wherein the two or more PPG sensors provide a higher signal to noise ratio as compared to any of the two or more PPG sensors individually; and
a processor configured to receive and process readings from the at least two PPG sensors.

2. The monitoring device of claim 1, further comprising an inertial measurement unit (IMU), wherein the processor processes the PPG readings in combination with the IMU.

3. The monitoring device of claim 1, further comprising one or more bandpass filters, wherein sensor information is passed through the one or more bandpass filters before being received by the processor.

4. The monitoring device of claim 1, wherein the band is elastic.

5. The monitoring device of claim 4, further comprising wires which operably connect the two or more PPG sensors, wherein the wires connecting the two or more PPG sensors are arranged on the band in a sinusoidal pattern.

6. The monitoring device of claim 1, wherein the processor is configured to transmit readings from the at least two PPG sensors to a mobile device.

7. The monitoring device of claim 1, wherein the processor is configured to transmit readings from the at least two PPG sensors to emergency services.

8. The monitoring device of claim 1, wherein the processor is a microcontroller.

9. A method of providing assistance to a subject based on a determined physiological condition of the subject, comprising:
determining that a physiological condition of the subject is abnormal based on information from two or more PPG sensors of a monitoring device worn on a wrist of the subject; and
transmitting an alert to emergency services indicating the abnormal physiological condition of the subject and a determined location of the subject.

10. The method of claim 9, wherein the physiological condition is the subject's heart rate.

11. The method of claim 9, wherein the alert is transmitted from the monitoring device via a mobile device.

12. The method of claim 9, wherein the monitoring device directly transmits the alert to emergency services.

13. The method of claim 9, wherein transmitting an alert to emergency services further comprises:
upon determining the abnormal condition, the monitoring device prompting the subject and waiting for a response therefrom; and
if the subject does not respond to the prompt within a predetermined amount of time, the monitoring device transmitting the alert to emergency services.

14. The method of claim 9, wherein determining that a physiological condition of the subject is abnormal further comprises:
continually monitoring a physiological condition to determine a value for a normal condition; and
provided the determined physiological condition has a value that varies from the normal condition value more than a predetermined amount, generating the alert.
